(19) United States
(12) Patent Application Publication
(10) Pub. No.: US 2015/0031930 A1
(43) Pub. Date: Jan. 29, 2015

(54) METHOD FOR ISOLATION OF CYMENE

(71) Applicant: HOLMEN AB, Stockholm (SE)

(72) Inventors: Jörg Brücher, Domsjo (SE); David Blomberg Saitton, Domsjo (SE)

(21) Appl. No.: 14/378,614

(22) PCT Filed: Feb. 14, 2013
(86) PCT No.: PCT/EP2013/052936

§ 371 Date: Aug. 13, 2014

Related U.S. Application Data
(60) Provisional application No. 61/598,545, filed on Feb. 14, 2012.

(30) Foreign Application Priority Data
Feb. 14, 2012 (SE) 1250118-5

(51) Int. Cl. C07C 7/00; B01D 3/34; B01D 3/00

(52) U.S. Cl. CPC C07C 7/005; B01D 3/009; B01D 3/34
USPC 585/807; 203/30

(57) ABSTRACT

The invention relates to a method of isolating cymene comprising the following steps: a) providing a solution comprising cymene and monoterpenes, b) adding sulphuric acid to the solution in a) such that the concentration of sulphuric acid in the solution is at least 0.5% (w/w), such as 1-25% (w/w), c) distilling the solution mixture from step b) such that a target chemical stream enriched in cymene is obtained and separated from a residual stream.

### TECHNICAL FIELD

The present invention relates to a method for isolation and purification of cymene. In particular it relates to a method for isolation of cymene from a solution comprising cymene and monoterpenes.

### BACKGROUND

Cymene is a naturally occurring aromatic organic compound which structure consists of a benzene ring substituted with a methyl group and an isopropyl group. The structure of cymene is similar to the numerous monoterpenes containing a cyclohexene or cyclohexadiene ring but in contrast to those and other monoterpenes, cymene is a stable compound not undergoing the typical reactions of terpenes. The most common geometric isomer is p-cymene, in which the alkyl groups are para-substituted. There also exists two less common geometric isomers; o-Cymene, in which the alkyl groups are ortho-substituted, and m-cymene, in which they are meta-substituted. p-cymene and m-cymene are valuable base chemicals which for example are used in fragrances, pharmaceuticals, herbicides, dyes, and heat transfer media. One other industrially important use of p-cymene is as a starting material for p-cresol production via the Hock-Lange synthesis pathway (1-4). Furthermore, p-cymene has also been proposed as a suitable ingredient in aviation fuel formulations. Compared to other aromatics used in automotive formulations, such as benzene, toluene or ethyl benzene, cymene has lower toxicity and is degraded easier in both aquatic and terrestrial systems.

Turpentine from boreal hard- and softwood species is a complex mixture of different monoterpenes with α-pinene, β-pinene and carene as main constituents. As sterical strained, unsaturated hydrocarbons, terpenes are highly reactive easily undergoing rearrangements, dimerisation or trimerisation or oxidation reactions. During the sulphate pulping process, terpenes stay unaltered and are condensed together with methanol from the off-gases. The turpentine is separated from other liquids by decantation, forming the typical crude sulphate turpentine (CST). Dominating impurities in CST are methanol along with organic sulphur compounds, polysulphides, and elementary sulphur. Turpentine is almost insoluble in water and thus CST and other turpentines generally contains only small amounts of water, such as less than 1%.

Monoterpenes are a class of terpenes that consist of two isoprene units and have the molecular formula C₁₀H₁₆. Monoterpenes may be linear (acyclic) or contain rings. Biochemical modifications such as oxidation or rearrangement produce the related monoterpenoids.

### SUMMARY OF THE PRESENT DISCLOSURE

Significant amounts of cymene are formed from the wood terpenes in sulphite pulping processes. The present inventors have also realized that cymene can be produced from other kinds of turpentine, such as for example crude sulphate turpentine. However, isolation of cymene from these kind of starting materials are challenging for several reasons. First of all, both the cymene formed in a sulphite pulping process and the cymene produced from e.g. crude sulphate turpentine generally comprises monoterpenes having a boiling point close to the boiling point of cymene and hence, a mixture of monoterpenes and cymene is hard to separate by conventional distillation processes. Furthermore, these kind of starting materials often comprise quite high concentrations of elementary sulphur (S₈), hydrogen sulphide (H₂S) and other sulphur compounds which further complicate the purification of cymene. For example, at elevated temperatures (and/or low absolute pressure) elementary sulphur can occur as volatile S₈ which can end up as contaminants in a target chemical stream from a distillation process. H₂S can also easily end up in the target chemical stream.

The present inventors have realized that there is a need for improved methods of isolation of cymene from a solution comprising cymene and monoterpenes. The inventors have solved this problem by designing a novel method for isolation of cymene. The method involves addition of sulphuric acid to a solution comprising cymene and monoterpenes such that the monoterpenes polymerizes into diterpenes, triterpenes and/or other oligoterpenes which have a boiling point considerably higher than the boiling point for cymene. Addition of sulphuric acid further promotes reaction of elementary sulphur, H₂S and other sulphur compounds into polysulphides having a high boiling point. Thereby the present invention provides a novel method where cymene easily can be separated from monoterpenes and sulphur compounds by a simple distilling process wherein the majority of the cymene ends up in the distillate and the majority of the monoterpenes and sulphur compounds remain in the residual fraction.

Thus a first aspect of the invention relates to a method of isolating cymene comprising the following steps:

a) providing a solution comprising cymene and monoterpenes,

b) adding sulphuric acid to the solution in a) such that the concentration of sulphuric acid in the solution is at least 0.5% (w/w), such as 1-25% (w/w),

c) distilling the solution mixture from step b) such that a target chemical stream enriched in cymene is obtained and separated from a residual stream.

### BRIEF DESCRIPTION OF THE FIGURES

FIG. 1 a shows a GC/MS chromatogram of a raw cymene fraction from a sulphite pulp mill comprising about 80% (w/w) cymene and 15% (w/w) monoterpenes. The Y-axis shows relative abundance and the X-axis shows the retention time in minutes.

FIG. 1 b shows a GC/MS chromatogram of the distillate from a distillation process of the raw cymene fraction shown in FIG. 1 a, wherein the distillation process was performed in the presence of about 6.2% (w/w) sulphuric acid. The Y-axis shows relative abundance and the X-axis shows the retention time in minutes.

FIG. 1 c shows a GC/MS chromatogram of the residual fraction from a distillation process of the raw cymene fraction shown in FIG. 1 a, wherein the distillation process was performed in the presence of about 6.2% (w/w) sulphuric acid. The Y-axis shows relative abundance and the X-axis shows the retention time in minutes.

### DETAILED DESCRIPTION

The present inventors have realized that it is hard to isolate cymene from a mixture of monoterpenes and cymene by conventional distillation processes, since the boiling point of cymene is close to the boiling point of most monoterpenes. For example the boiling point of cymene is about 177° C. and the boiling points of carene, alpha-pinene and beta-pinene, which are the most common monoterpenes present in turpentine, are about 169° C., 157° C. and 167° C. respectively. The present invention relates to a novel method which solves this problem.

A first aspect of the invention relates to a method of isolating cymene comprising the following steps:
a) providing a solution comprising cymene and monoterpenes,
b) adding sulphuric acid to the solution in a) such that the concentration of sulphuric acid in the solution is at least 0.5% (w/w), such as 1-25% (w/w),
c) distilling the solution mixture from step b) such that a target chemical stream enriched in cymene is obtained and separated from a residual stream.

Addition of sulphuric acid to a solution comprising cymene and monoterpenes leads to polymerization of monoterpenes into oligomers (e.g. diterpene and triterpene) having a boiling point which is considerably higher that the boiling point for cymene, such as at least 50° C. higher or at least 100° C. higher.

Even though a sulphuric acid concentration of 0.5% (w/w) is sufficient for polymerization of at least part of the monoterpenes, higher concentrations are usually preferred to increase the grade of purification. Therefore, in a preferred embodiment sulphuric acid is added to a concentration above 2% (w/w) more preferably above 3% (w/w) and most preferably above 5% (w/w) in step b).

In one embodiment the target chemical stream obtained in step c) comprises at least 75%, such as at least 80%, such as at least 90%, of the cymene present in the solution comprising cymene and monoterpenes provided in step a). In one embodiment at least part of the monoterpenes from step a) react to diterpenes, triterpenes, oligoterpenes and/or polyterpenes in step b) and/or c). In a preferred embodiment the majority, such as at least 50%, preferably at least 90% of the terpenes in the residual stream in step c) is in the form of diterpenes, triterpenes, oligoterpenes and/or polyterpenes. In one embodiment at least 50%, preferably at least 90% of the terpenes in the solution in step a) is in the form of monoterpenes.

It is possible that a portion of the monoterpenes present in the solution obtained in step a) is oxidized into cymene during step b) and or c). However, since the monoterpenes are converted into oligomers during step b) and/or c) the levels of produced cymene during step b) and/or c) will normally be small or even negligible. Therefore, in one embodiment at least 50%, such as at least 75%, preferably at least 90% such as at least 99% such as all of the cymene molecules present in the target chemical stream obtained in step c) is the same cymene molecules as the cymene molecules present in the solution in step a).

Several industrial relevant sources of cymene comprise not only terpenes but also quite high concentrations of sulphur compounds such as elementary sulphur (S₈) or hydrogen sulphide (H₂S). In conventional distillation processes, the sulphur contaminations further complicates the purification of cymene from these kinds of starting materials. However, the method according to the present invention solves this problem and thus the method is particularly suitable for starting materials having a relatively high concentration of sulphur. Accordingly, in one embodiment the solution comprising cymene and monoterpenes provided in step a) comprises elementary sulphur and/or H₂S and at least part of the elementary sulphur and/or H₂S reacts to form polysulphides in step b) and/or step c). In one embodiment the sulphur content of the solution comprising cymene and monoterpenes provided in step a) is at least 0.1% (w/w), such as at least 0.5% (w/w). In one embodiment the solution comprising cymene and monoterpenes provided in step a) is turpentine and in one embodiment the turpentine is crude sulphate turpentine (CST) or turpentine from a sulphite pulping process (sulphite turpentine) or turpentine from a thermo mechanical pulping process (TMP turpentine). CST and sulphite turpentine generally comprise high levels of sulphur and hence the method is particularly suitable for these kinds of turpentine. Hence in a preferred embodiment the solution comprising cymene and monoterpenes provided in step a) is derived from CST or sulphite turpentine. In one embodiment the solution comprising cymene and monoterpenes provided in step a) is sulphite turpentine

The present inventors have also realized that cymene can be produced from a solution comprising cyclic monoterpenes. Said method comprises the following steps:

i) providing a solution comprising cyclic monoterpenes having the formula C₁₀H₁₆,

ii) addition of a catalyst comprising Fe³⁺ and/or Fe²⁺ ions,

iii) addition of sulphur dioxide to the solution,

iv) incubation of the solution mixture from step iii) for a reaction time at a reaction temperature such that at least part of the cyclic monoterpenes are oxidized to cymene such that a solution comprising cymene is obtained.

Therefore, in one embodiment the solution comprising cymene and monoterpenes provided in step a) is provided by a method comprising the steps of:

i) providing a solution comprising cyclic monoterpenes having the formula C₁₀H₁₆,

ii) addition of a catalyst comprising Fe³⁺ and/or Fe²⁺ ions,

iii) addition of sulphur dioxide to the solution,

iv) incubation of the solution mixture from step iii) for a reaction time at a reaction temperature such that at least part of the cyclic monoterpenes are oxidized to cymene such that a solution comprising cymene is obtained. In one embodiment the solution comprising cyclic monoterpenes having the formula C₁₀H₁₆ is CST.

Sometimes it is preferred to perform the distilling process at a lower temperature. This can be achieved if the pressure is decreased in the distilling process. Thus, in one embodiment the distilling in step c) is performed at a distilling temperature below 170° C., such as below 120° C., such as 20° C.-120° C. In one embodiment the distilling in step c) is performed at a reduced pressure, such as at a pressure below 101 kPa, such as at a pressure between 1-50 kPA such as at a pressure between 5-10 kPa.

The cymene in the target chemical stream obtained in step c) may contain water and small amounts of sulphur compounds. The cymene in the target chemical stream can be further purified by removal of water and sulphur contents. This can be achieved by addition of a dehydrating agent. Therefore, in one embodiment the method further comprises a step d) removing water from the target chemical stream obtained in c) by addition of a dehydrating agent. In one embodiment the dehydration agent is selected from a substance comprising CaO, anhydrous MgSO₄, anhydrous Na₂SO₄ and/or anhydrous CaCl₂. In a preferred embodiment the dehydration agent comprises CaO. In one embodiment the CaO in added to a concentration of 1-25% (w/w), such as 2-10% (w/w).

In one embodiment at least part of the monoterpenes in step a) is a cyclic monoterpene. In one embodiment at least part of the terpenes in step a) is α-pinene, β-pinene, carene, sabinene, α-thujene, β-thujene and/or limonene. Turpentine from boreal hard- and softwood species is a complex mixture of different terpenes with α-pinene, β-pinene and carene as main constituents. Therefore, in a preferred embodiment at least part of the monoterpenes in step a) is α-pinene, β-pinene, and/or carene, and in a most preferred embodiment the majority of the monoterpenes in step a) are α-pinene, β-pinene and/or carene. In one embodiment at least part of the cymene in step a) is p-cymene.

The polymerized monoterpens have a high viscosity which gives the obtained residual stream a tar-like appearance and makes it solid at room temperature. If the concentration of monoterpens in the solution comprising cymene and monoterpene obtained in step a) is too high, cymene can get stuck in the formed tar-like substance during step b) and or c). Thus, in a preferred embodiment the concentration of monoterpenes in the solution comprising cymene and monoterpenes provided in step a) is below 70% (w/w), preferably below 50% (w/w). In another embodiment the concentration of cymene in the solution comprising cymene and monoterpenes provided in step a) is at least 30% (w/w), preferably at least 50% (w/w).

### EXAMPLES

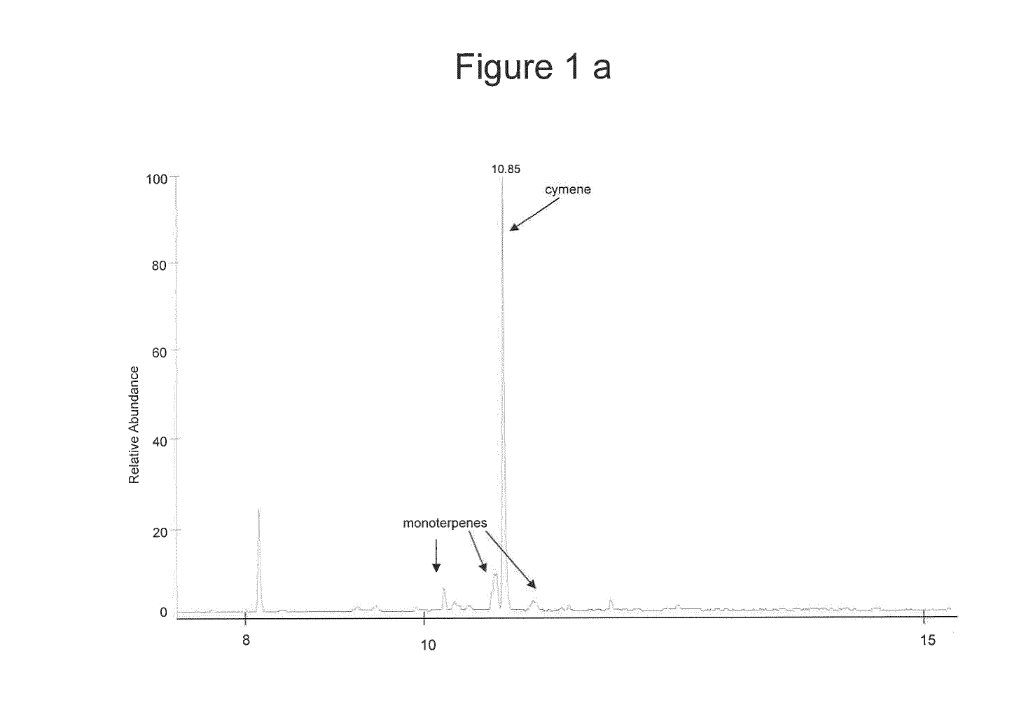

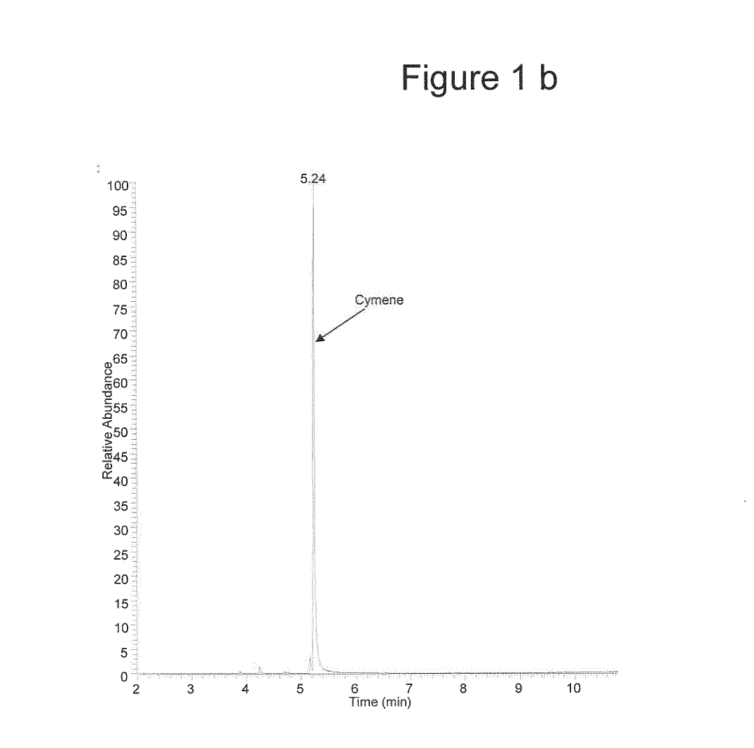

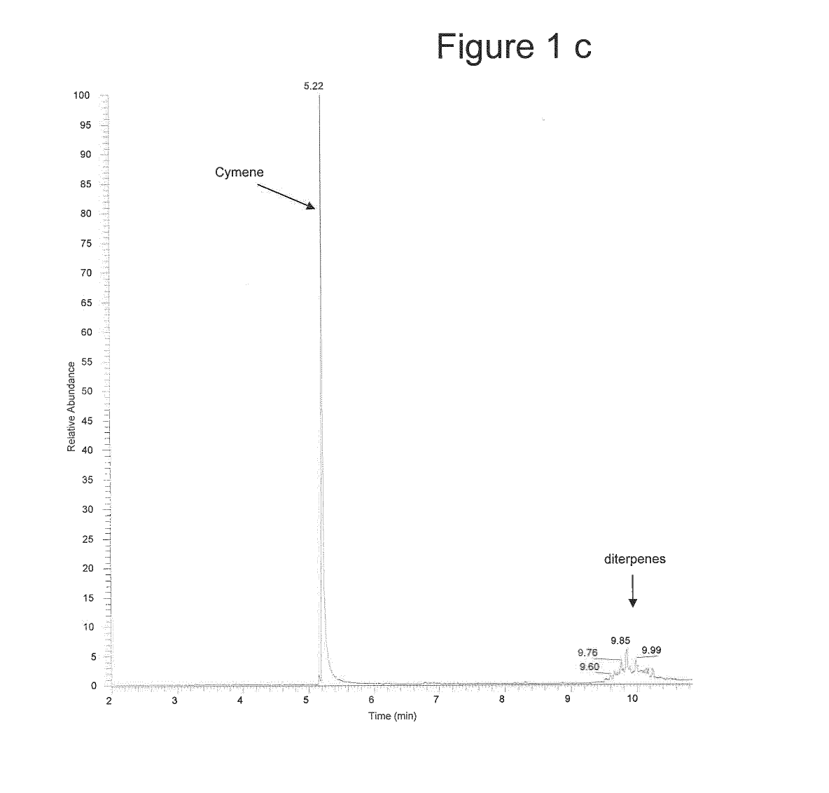

100 g of sulphuric acid was added to 1.5 litres of raw cymene, obtained form a sulphite pulp mill. This corresponds to a sulphuric acid concentration of about 6.2% (w/w). The raw cymene comprised 80% (w/w) cymene 15% (w/w) monoterpenes, 2% (w/w) water and a total sulphur content of 3% (w/w). The temperature of the resulting raw cymene/sulphuric acid mixture spontaneously raised to about 75° C. and the mixture was thereafter further heated to 110° C. for 10 minutes. After cooling to 70° C., the mixture was distilled at a pressure of 50 hPa, using a 60 cm Vigreux column. After this distillation the cymene concentration in the obtained distillate was 98% (w/w) and the yield in distillation process was 80%). The concentration of contaminating monoterpenes in the distillate was 1.4% (w/w). To remove traces of water and sulphur, the distillate was further treated with 5% (w/w) calcium oxide (CaO). After this treatment the total sulphur content was 0.06% (w/w) and the water content was 0.01% (w/w). Chromatograms of the raw cymene, the distillate and the residual fraction are shown in FIG. 1. As evident from these chromatograms, the majority of the terpenes present in the raw cymene are in the form of monoterpenes whereas the majority of the terpenes in the residual fraction are in the form of diterpenes.

Measurements of cymene, monoterpenes sulphur and water concentrations were preformed by MoRe Research AB, Sweden. The concentrations of cymene and monoterpenes were measured by GC/MS; the water concentration was measured using Karl Fischer titration and the concentration of total sulphur was measured using SCAN CM 57-99 (MoRe Research AB), a method based on Schöniger combustion and wherein the end-determination is based on ion chromatography.

### REFERENCES

1. Ito et al., Hydrocarb. Process. 52 (8) (1973), 89.

2. Welstead et al., Encyclopedia Chem. Technol. 9 (1978), 544.

3. Derfer et al., Encyclopedia Chem. Technol. 22 (1978), 709.

4. Barman et al., Chemical Engineering Journal 114 (2005).

1.-14. (canceled)

15. A method of isolating cymene comprising:
a) providing a solution comprising cymene and monoterpenes,
b) adding sulphuric acid to the solution in a) such that the concentration of sulphuric acid in the solution is at least 0.5% (w/w),
c) distilling the solution mixture from b) such that a target chemical stream enriched in cymene is obtained and separated from a residual stream.

16. The method according to claim 15, the concentration of sulphuric acid in the solution is 1-25% (w/w).

17. The method according to claim 15, the concentration of sulphuric acid in the solution is 3-25%.

18. The method according to claim 15, wherein at least part of the monoterpenes from step a) react to one of the following selected from diterpenes, triterpenes, oligoterpenes and polyterpenes in one or both of b) and c).

19. The method according to claim 15, wherein the majority of the terpenes in the residual stream in c) is in the form of one of the following selected from diterpenes, triterpenes, oligoterpenes and polyterpenes.

20. The method according to claim 19, wherein at least 50% of the terpenes in the residual stream in c) is in the form of one of the following selected from diterpenes, triterpenes, oligoterpenes and polyterpenes.

21. The method according to claim 19, wherein at least 90% of the terpenes in the residual stream in c) is in the form of one of the following selected from diterpenes, triterpenes, oligoterpenes and polyterpenes.

22. The method according to claim 15, wherein the majority of the terpenes in the solution in a) is in the form of monoterpenes.

23. The method according to claim 22, wherein the at least 50% of the terpenes in the solution in a) is in the form of monoterpenes.

24. The method according to claim 22, wherein the at least 90% of the terpenes in the solution in a) is in the form of monoterpenes.

25. The method according to claim 15, wherein the distilling in c) is performed at a reduced pressure.

26. The method according to claim 25, wherein the distilling in c) is performed at a pressure below 101 KPa.

27. The method according to claim 25, wherein the distilling in c) is performed at a pressure between 1-50 kPA.

28. The method according to claim 25, wherein the distilling in c) is performed at a pressure between 5-10 KPa

29. The method according to claim 15, wherein the distilling in c) is performed at a distilling temperature below 170° C.

30. The method according to claim 29, wherein the distilling in c) is performed at a distilling temperature below 120° C.

31. The method according to claim 15, further comprising a removing water from the target chemical stream obtained in c) by addition of a dehydrating agent.

32. The method according to claim 31, wherein the dehydration agent is selected from a substance comprising CaO, anhydrous MgS0₄, anhydrous Na₂S0₄ and anhydrous CaCl₂.

33. The method according to claim 32, wherein the dehydration agent comprises CaO.

34. The method according to claim 33, wherein the CaO in added to a concentration of 1-25% (w/w).

35. The method according to claim 33, wherein the CaO in added to a concentration of 2-10% (w/w).

36. The method according to claim 15, wherein the solution in a) comprises one selected from elementary sulphur and H₂S, and wherein at least part of the elementary sulphur and/or H₂S reacts to form polysulphides in one or both of b) and c).

37. The method according to claim 15, wherein the solution comprising cymene and monoterpenes provided in a) has a sulphur content above 0.1% (w/w).

38. The method according to claim 15, wherein the concentration of cymene in the solution comprising cymene and monoterpenes provided in a) is at least 30% (w/w).

39. The method according to claim 38, wherein the concentration of cymene in the solution comprising cymene and monoterpenes provided in a) is at least 50% (w/w).

40. The method according to claim 15, wherein the concentration of monoterpenes in the solution comprising cymene and monoterpenes provided in a) is below 70% (w/w).

41. The method according to claim 40, wherein the concentration of monoterpenes in the solution comprising cymene and monoterpenes provided in a) is below 50% (w/w).
